(19) United States
(12) Patent Application Publication
(10) Pub. No.: US 2019/0201232 A1
(43) Pub. Date: Jul. 4, 2019

(54) AIRWAY EXTENSION DEVICE

(71) Applicant: Donghwan KIM, Seoul (KR)

(72) Inventor: Donghwan KIM, Seoul (KR)

(21) Appl. No.: 15/964,044

(22) Filed: Apr. 26, 2018

(30) Foreign Application Priority Data
Dec. 29, 2017 (KR) 10-2017-0183882

(51) Int. Cl. A61F 5/56

(52) U.S. Cl. CPC A61F 5/566

(57) ABSTRACT

Disclosed herein is an airway extension device. In particular, the airway extension device includes: an upper teeth seating portion located on an upper side and seated with a plurality of upper teeth; a lower teeth seating portion located on a lower side and seated with a plurality of lower teeth; and at least one negative pressure forming hole formed on a spaced part corresponding to a spaced location between upper and lower teeth generated after the airway extension device that a wearer wears is mounted in an oral cavity so as to penetrate in a vertical direction, thereby improving snoring or sleep apnea of the wearer.

### CROSS-REFERENCE TO RELATED APPLICATION(S)

This application claims the benefit of and priority to Korean Patent Application No. 10-2017-0183882, filed on Dec. 29, 2017, the content of which is incorporated by reference in its entirety.

### TECHNICAL FIELD

The present disclosure relates to an airway extension device.

### BACKGROUND

In general, snoring and sleep apnea are caused by narrowing an air passage (airway) during the sleep. An air passes through soft parts of a body, such as a palate, a tongue, and a uvula at each breathe. Since these muscles are sufficiently tense during the daytime, the airway is secured and thus a sound rarely occurs by breathing. However, the case in which the muscles are relaxed during the sleep and the air passage is narrow frequently occurs. When the airway is narrow, an air velocity of inhalation or exhalation increases to generate a vibration, such that snoring is caused.

On the other hand, an air passage of a patient with severe snoring may temporarily obstructed completely, which is called sleep apnea.

A variety of structures for treating and improving the snoring and sleep apnea are known in the art.

Some of the known wearable snoring preventing structures are configured to cover a mouth by sealing upper and lower lips and the others produce a reaction when air passes through a uvula and a soft palate during the sleep to recognize vibrating these tissue portions and significantly reduce an air volume passing through the mouth without completely covering the mouth, thereby minimizing the vibration in response to the moving velocity of air as described above.

### SUMMARY

An objective of embodiments disclosed herein is to provide an airway extension device capable of preventing or reducing snoring during the sleep or improving sleep apnea.

Another objective of the embodiments disclosed herein is to provide a personalized airway extension device for a wearer.

Still another objective of the embodiments disclosed herein is to provide an airway extension device capable of easily detachably mounted for a wearer and increasing accessibility of a negative pressure formation by a tongue in an oral cavity and a lower lip outside the oral cavity.

Objectives of the embodiments disclosed herein are not limited to the above-mentioned objectives. That is, other objectives that are not mentioned may be obviously understood by those skilled in the art.

According to an embodiment disclosed herein, an airway extension device includes: an upper teeth seating portion located on an upper side and seated with a plurality of upper teeth; a lower teeth seating portion located on a lower side and seated with a plurality of lower teeth; and at least one negative pressure forming hole formed on a spaced part corresponding to a spaced location between upper and lower teeth generated after the airway extension device that a wearer wears is mounted in an oral cavity so as to penetrate in a vertical direction.

According to another embodiment disclosed herein, an airway extension device includes: an upper teeth seating portion located on an upper side and seated with a plurality of upper teeth; a lower teeth seating portion located on a lower side and seated with a plurality of lower teeth; a teeth partition surface formed to partition the upper teeth seating portion and the lower teeth seating portion; and at least one negative pressure forming hole formed on the teeth partition surface and formed on a spaced part corresponding to a spaced location between upper and lower teeth generated after the airway extension device that a wearer wears is mounted in an oral cavity to penetrate in a vertical direction.

The at least one negative pressure forming hole may be formed between an upper line of tooth point formed by connecting lower ends of the plurality of upper teeth and a lower line of tooth point formed by connecting upper ends of the plurality of lower teeth.

The airway extension device may further include: a front rim portion provided on a front end side of the upper teeth seating portion and having an inner side surface surrounding an outer side surface of the plurality of upper teeth and an outer side surface provided to support an inner side surface of an upper lip.

The at least one negative pressure forming hole may form a negative pressure of a predetermined pressure between a tongue located inside an oral cavity and a lower lip located outside thereof, having the front rim portion interposed therebetween.

The at least one negative pressure forming hole may form a negative pressure at a pressure enough to adhere a tongue located inside an oral cavity having the front rim portion interposed therebetween to a palate.

The negative pressure forming hole may include at least one central hole which is formed at a central side of the plurality of upper teeth and the plurality of lower teeth.

The negative pressure forming hole may further include at least one side hole each symmetrically formed on left and right sides of the at least one central hole.

The airway extension device may further include: a tongue seating surface seated with an edge part of a tongue while forming an inner side surface of the lower teeth seating portion and partitioning between the plurality of lower teeth, wherein the at least one negative pressure forming hole is formed at a boundary of the tongue seating surface adjacent to the upper teeth seating portion.

The airway extension device may further include: left and right side rim portions formed on left and right sides of a tongue seating surface while protruding upward to be partitioned from the lower end part of the upper teeth provided on a side part among the plurality of upper teeth, wherein the at least one negative pressure forming hole is formed under upper ends of the left and right side rim portions.

The at least one negative pressure forming hole may be provided to be closed at the inside and outside of the oval cavity due to the nature of a nervous reaction of the tongue or the lower lip and may be formed on a location where the lower surface of the tip edge part of the tongue located inside the oval cavity closes the negative pressure forming hole while covering the upper side thereof to penetrate in the vertical direction.

The airway extension device may further include: a sealing portion provided on a part of a portion contacting an inside of a wearer's oval cavity to block a direct contact with the inside of the oval cavity.

The airway extension device may include: the sealing portion includes: a first sealing portion provided on an edge end contacting the wearer's lower lip; and a second sealing portion provided on an edge part of a tongue seating surface on which the wearer's tongue part is seated.

### BRIEF DESCRIPTION OF THE DRAWINGS

FIG. 1 is a perspective view of an airway extension device according to an embodiment disclosed herein.

FIG. 2 is a plan view of FIG. 1.

FIG. 3 is a bottom view of FIG. 1.

FIG. 4 is a front view of FIG. 1.

FIG. 5 is a rear view of FIG. 1.

FIGS. 6A and 6B are conceptual views showing locations of a tongue in an example of applying the airway extension device according to an embodiment disclosed herein and an example of the related art.

FIGS. 7A, 7B, 8A, 8B, 9A, 9B, 10A and 10B are plan views and bottom views of the airway extension device according to various embodiments disclosed herein.

FIG. 11 is a configuration view showing a sealing portion of the airway extension device according to an embodiment disclosed herein.

### DETAILED DESCRIPTION OF EMBODIMENTS

Hereinafter, exemplary embodiments will be described with reference to the accompanying drawings. It is to be noted that in giving reference numerals to components of each of the accompanying drawings, the same components will be denoted by the same reference numerals even though they are shown in different drawings. Further, in describing exemplary embodiments, well-known constructions or functions will not be described in detail in the case in which it is decided that they may unnecessarily obscure the understanding of the present disclosure.

In describing components of the embodiments disclosed herein, terms such as upper, lower, left, and right may be used. These terms are merely for distinguishing directions of the components, and should not be limited to being substantially indicative of the directions. Unless indicated otherwise, all the terms used in the present specification, including technical or scientific terms, have the same meanings as meanings that are generally understood by those skilled in the art to which the present disclosure pertains. It must be understood that the terms defined by the dictionary generally used are identical with the meanings within the context of the related art, and they should not be ideally or excessively formally defined unless the context clearly dictates otherwise.

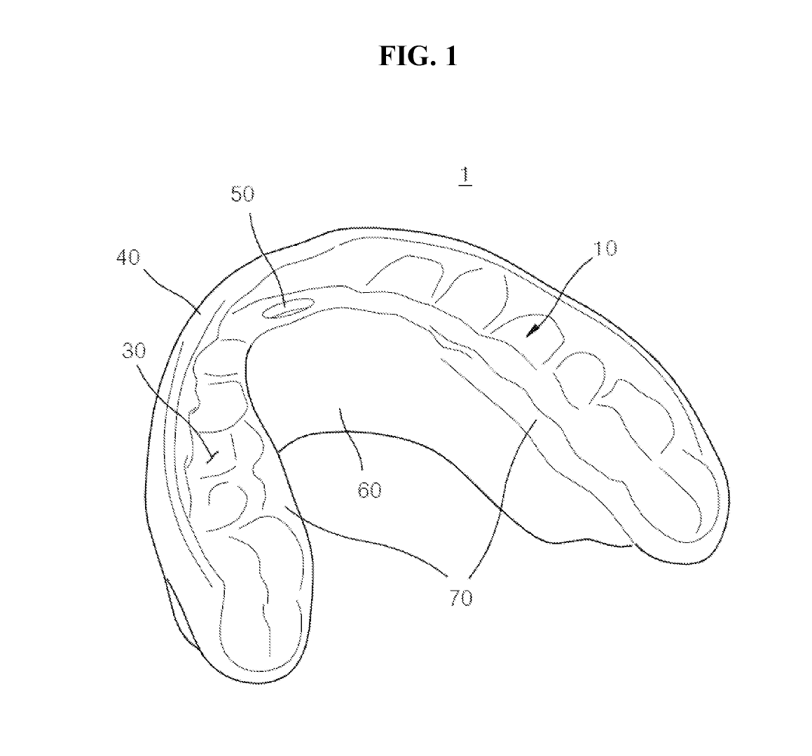

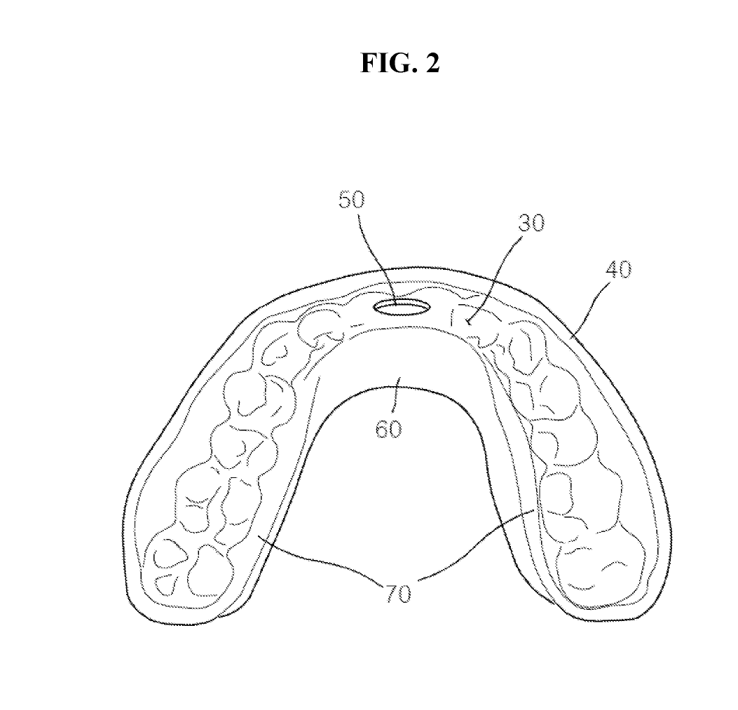

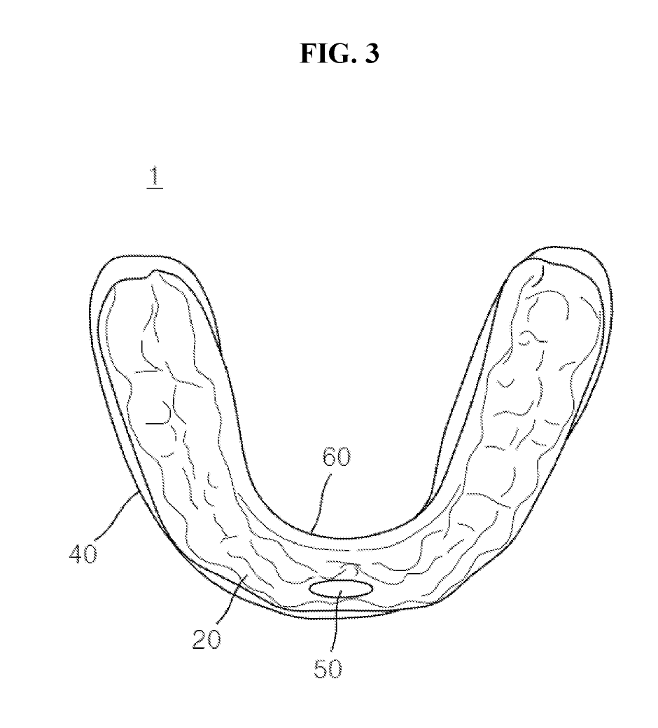

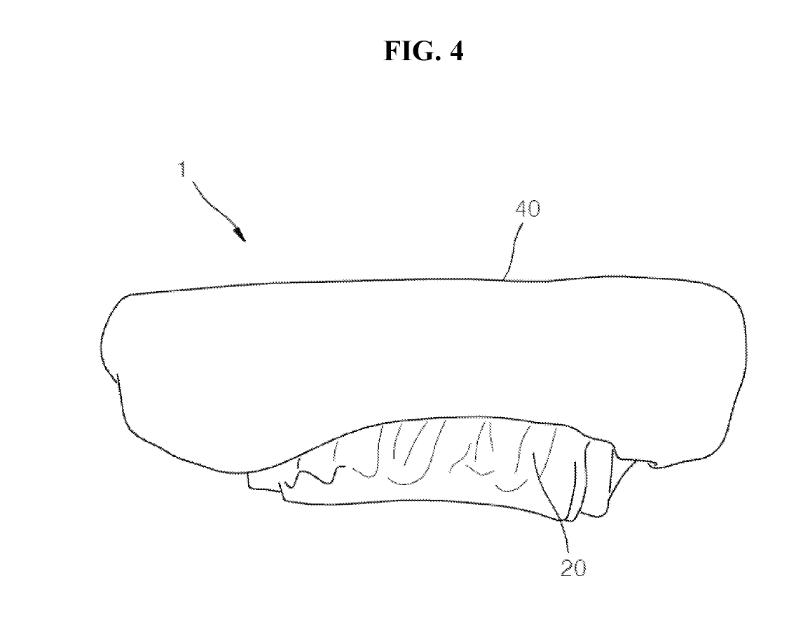

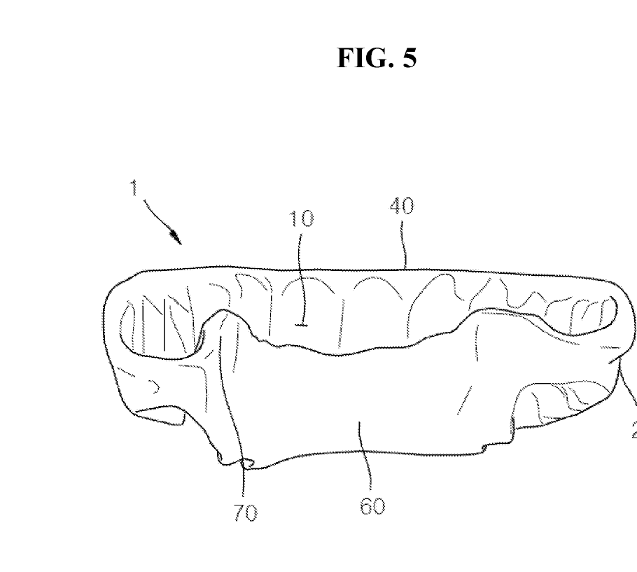

FIG. 1 is a perspective view of an airway extension device according to an embodiment disclosed herein, FIG. 2 is a plan view of FIG. 1, FIG. 3 is a bottom view of FIG. 1, FIG. 4 is a front view of FIG. 1, and FIG. 5 is a rear view of FIG. 1.

As shown in FIGS. 1 to 5, an embodiment of an airway extension device 1 includes an upper teeth seating portion 10 located on an upper side and seated with a plurality of upper teeth, a lower teeth seating portion 20 located on a lower side and seated with a plurality of lower teeth, and at least one negative pressure forming hole 50 formed on a spaced part of the airway extension device corresponding to a spaced location between upper and lower teeth as a location at which the negative pressure is generated when the airway extension device that a patient (hereinafter, referring to as ‘wearer’) wears is mounted in an oral cavity so as to penetrate in a vertical direction.

The upper teeth seating portion 10 includes upper teeth seating grooves 30 on which a plurality of upper teeth of a wearer are seated when the wearer mounts the airway extension device 1 according to the embodiment disclosed herein into the oral cavity.

Since the upper teeth seating grooves 30 are manufactured by cutting out a pattern of upper teeth of a wearer in advance so as to fit the upper teeth of the wearer, they cannot be mounted on other wearers and may have a shape to fit only the airway extension device 1 according to the present disclosure provided in a single number.

The lower teeth seating portion 20 is a portion on which an inner side surface of a plurality of lower teeth of a wearer is seated when the wearer mounts the airway extension device 1 according to the embodiment disclosed herein into the oral cavity.

The lower teeth seating portion 20 is also manufactured by being patterned to fit a wearer simultaneously with the upper teeth seating grooves 30 formed on the upper teeth seating portion 10, and as a result may have a shape in which it is fitted with the inner side surface of a plurality of lower teeth of a wearer.

On the other hand, the negative pressure forming hole 50 is closed by wearer's body parts (in particular, the tongue in the oral cavity and the lower lip on the outside of the mouth) when the user wears the airway extension device 1 according to the embodiment disclosed herein into the oral cavity to serve to form a predetermined negative pressure within the oral cavity by inhalation or exhalation while a wearer breathes through his/her nose.

When the negative pressure is formed within the oral cavity by the negative pressure forming hole 50, the wearer's tongue raises a hyoid bone (not shown) provided with a tongue's muscle while moving upward forward to extend an air passage (airway) inside the oral cavity. A detailed description thereof will be described later in detail.

Generally, a plurality of upper teeth protrude outward relative to a plurality of lower teeth when an unspecified number of wearers close normally their mouth.

In particular, the airway extension device 1 according to the embodiment disclosed herein is manufactured by being stuck to a wearer's mouth at the time of manufacturing and cutting out a pattern to fit a tooth shape of a wearer. Here, the negative pressure forming hole 50 may be formed on the spaced part between inside and outside of a plurality of upper teeth and a plurality of lower teeth when being manufactured based on a mold produced from a normal biting posture of a wearer, not based on a mold produced from an irregular biting posture of a wearer to penetrate in the vertical direction.

In the airway extension device 1 according to the embodiment in which the negative pressure forming hole 50 is formed, when a wearer take sleep while mounting the airway extension device 1 in his or her mouth, a wearer's tongue finds and blocks the negative pressure forming hole 50 by a nature of a nerve reaction to block heterogeneous parts within an oral cavity and blocks the negative pressure forming hole 50 within the oral cavity while a tip of the tongue moves upward forward within the oral cavity. At the same time, a wearer's lower lip may also move so that the negative pressure forming hole 50 exposed to the outside is blocked at the outside of the oral cavity.

In particular, in the airway extension device 1 according to the embodiment of disclosed herein, since the negative pressure forming hole 50 is provided to penetrate in the vertical direction, generally, a wearer's tongue tip does not block the negative pressure forming hole 50 but moves so that a lower edge end part of the tongue covers the negative pressure forming hole 50, such that a wearer's tongue further moves upward forward. According to this logic, although not shown in the drawings, the case in which the negative pressure forming hole 50 is formed to penetrate in a horizontal direction unlike the embodiment disclosed herein is less effective than the case in which the wearer's tongue tip simply moves to block the negative pressure forming hole 50.

As described above, the negative pressure forming hole 50 among the components of the airway extension device 1 according to the embodiment induces a wearer's tongue to be seated while being located at an optimum location to extend the air passage (airway) in the oral cavity in a sleep state, thereby helping improve the snoring or sleep apnea of the wearer.

Meanwhile, referring to FIGS. 1 to 5, the airway extension device 1 according to the embodiment disclosed herein may further include a teeth partition surface (not represented by a reference symbol) formed to partition the upper teeth seating portion 10 and the lower teeth seating portion 20.

The teeth partition surface may be defined as an integral structure including the upper teeth seating portion 10 and the lower teeth seating portion 20. That is, the above-described upper teeth seating portion 10 is formed on the inner side surface of the teeth partition surface to fit the shape of the wearer's upper teeth, and the above-described lower teeth seating portion 20 may be formed on the outer side surface of the teeth partition surface to fit the shape of the inner side surface of the wearer's lower teeth.

Therefore, it should be understood that the teeth partition surface is formed as the whole tooth shape since it is manufactured by cutting out a pattern of the inside of the wearer's oral cavity.

Here, at least one negative pressure forming hole 50 is formed on the teeth partition surface and may be formed on the spaced part corresponding to the spaced location between the upper teeth and the lower teeth generated after the wearer mounts in the oral cavity to penetrate in the vertical direction.

More specifically, when an arbitrary connecting line formed by connecting between the lower ends of the plurality of upper teeth of the wearer is defined as an ‘upper line of tooth point’ and an arbitrary connecting line formed by connecting the upper ends of the plurality of lower teeth of the wearer is defined as a ‘lower line of tooth point’, the above-described at least one negative pressure forming hole 50 may be formed between the upper line of tooth point and the lower line of tooth point.

In addition, referring to FIG. 2, the airway extension device 1 according to the embodiment disclosed herein is provided on the front end side of the upper teeth seating portion 10, and may further include a front rim portion 40 provided to have an outer side surface to support an inner side surface of an upper lip while the inner side surface surrounding the outer side surface of the plurality of upper teeth.

When the wearer mounts a calibrating device according to the embodiment of disclosed herein by the front rim portion 40, the plurality of upper teeth are closed without being exposed forward except for an upper gum. The outer side surface of the front rim portion 40 may be arranged to cover the wearer's upper lip.

Meanwhile, referring to FIGS. 1 to 5, the airway extension device 1 according to the embodiment disclosed herein may further include a tongue seating surface 60 partitioning between the plurality of lower teeth by seating the edge part of the tongue while forming the inner side surface of the lower teeth seating portion 20.

Here, the tongue seating surface 60 has not a component separately partitioned like the above-described teeth partition surface, but may be defined as the inner side surface opposite to the outer side part provided with the lower teeth seating portion 20, and any part on which a lower surface of the edge part of the tongue is seated may be formed as the tongue seating surface 60.

The negative pressure forming hole 50 may be formed at a boundary of the tongue seating surface 60 adjacent to the upper teeth seating portion 10.

Referring to FIGS. 1 to 5, the airway extension device 1 according to the embodiment disclosed herein may further include left and right side rim portions 70 which are formed on left and right sides of the tongue seating surface 60 while protruding upward to be partitioned from the lower end part of the upper teeth provided on the side part among the plurality of upper teeth.

The left and right side rim portions 70 may protrude upward to partition the tongue seating surface 60 from the upper teeth seating portion 10.

Here, the negative pressure forming hole 50 may be formed under the upper ends of the left and right side rim portions 70.

At least one negative pressure forming hole 50 thus formed may form a negative pressure of a predetermined pressure between the tongue located inside the oral cavity and the lower lip located outside the oral cavity, having the front rim portion 40 interposed therebetween.

The negative pressure of the predetermined pressure is enough to adhere the tongue located inside the oral cavity to the palate, having the front rim portion 40 interposed therebetween. The tip of the tongue is located to be fully stuffed in the upper side which is the palate side while being seated on the tongue seating surface 60 to be described later, thereby forming the optimum negative pressure within the oral cavity.

That is, at least one negative pressure forming hole 50 may be provided to be closed at the inside and outside of the oval cavity due to a nature of a nervous reaction of the tongue or the lower lip, and may be formed at a location where the lower surface of the tip edge part of the tongue located inside the oval cavity closes the negative pressure forming hole 50 while covering the upper side thereof to penetrate in the vertical direction.

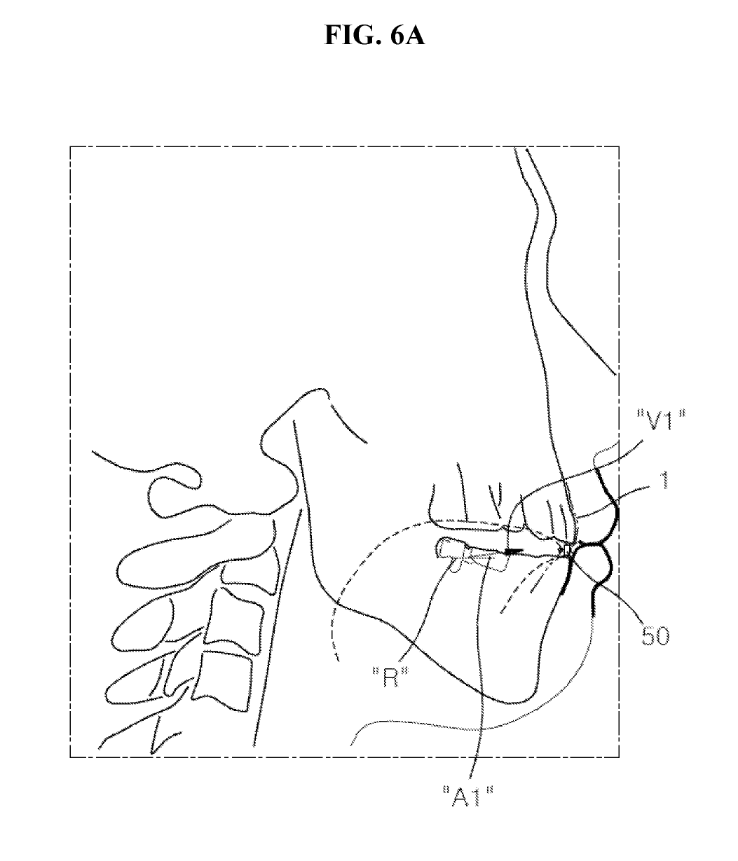

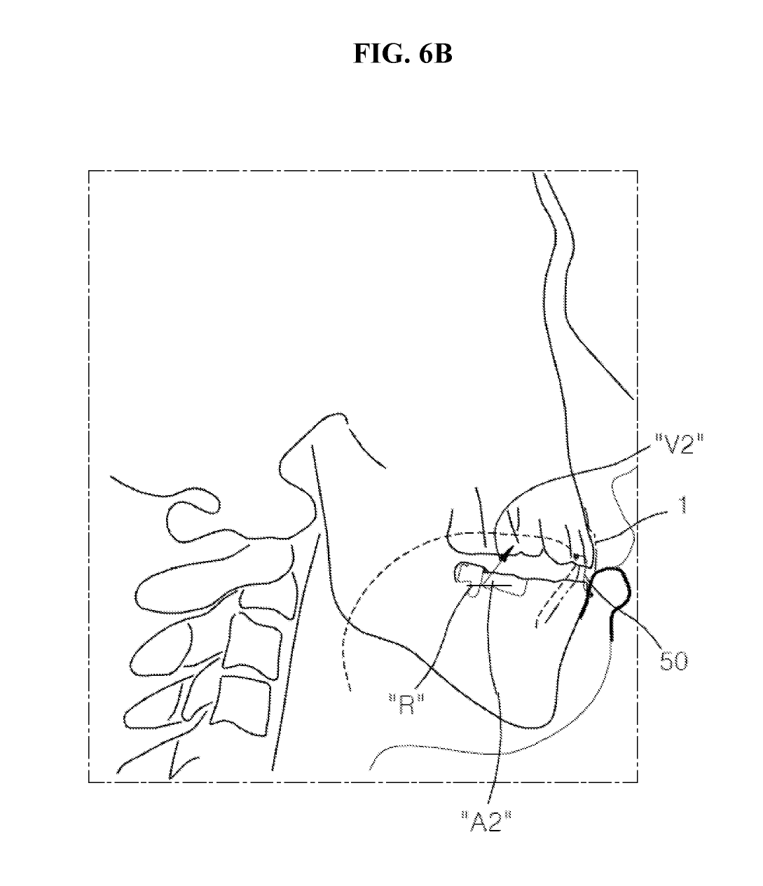

FIGS. 6A and 6B are conceptual views showing locations of a tongue in an example of applying the airway extension device according to an embodiment disclosed herein and an example of the related art.

As shown in FIG. 6A, the case in which the above-described negative pressure forming hole 50 is provided to penetrate in the horizontal direction, and the case in which the negative pressure-forming hole 50 is provided to penetrate in the vertical direction as shown in FIG. 6B will be comparatively described as follows.

First, referring to FIG. 6A, the outside of the negative pressure forming hole 50 is blocked by the wearer's lower lip, and the inside of the negative pressure forming hole 50 is blocked by an operation of contacting the tip of the tongue of the wearer therewith, thereby forming the negative pressure within the wearer's oral cavity.

However, the negative pressure forming hole 50 as shown in FIG. 6A is formed in the horizontal direction, and only the tip of the wearer's tongue may be blocked by contacting the inside of the negative pressure forming hole 50. At this time, referring to the movement of the wearer's tongue, as shown in FIG. 6A, an outwardly upwardly inclined force vector V1 is formed to have a forming angle of approximately “A1” with respect to an arbitrary reference line R. Here, a distance of the upper surface of the tongue floating toward the palate side within the oral cavity cannot but be smaller than in the case described with respect to FIG. 6B.

That is, referring to FIG. 6B, the inside of the negative pressure forming hole 50 formed to penetrate in the vertical direction is blocked by being covered by the bottom edge portion of the tip of the tongue rather than the tip of the wearer's tongue. At this time, referring to the movement of the wearer's tongue, as shown in FIG. 6B, an outwardly upwardly inclined force vector V2 is formed to have a forming angle “A2” which is an angle larger than the above-described “A1” with respect to the arbitrary reference line R. Therefore, the upper surface of the tongue further floats toward the palate on the whole in the case in which the negative pressure forming hole 50 is formed in the vertical direction at the spaced parts of a plurality of the upper teeth and the lower teeth than in the case in which simply the negative pressure forming hole 50 is formed in the horizontal direction.

The difference in the movement of the tongue for blocking the negative pressure forming holes 50 differently formed as described above within the oral cavity causes the difference in the whole moving distance of the above-described hyoid bone and there is an advantage in that the airway of at least 2 mm can be additionally secured.

Additionally securing of the air passage (airway) of 2 mm may have an effect of reducing an air flow rate to such an extent that the wearer's symptoms such as the snoring, the sleep apnea, the teeth grinding, and the clenching may be alleviated.

In addition, the negative pressure forming hole 50 may act as a breathable space for mouth breathing if the wearer's nose is completely obstructed.

In addition, the wearer's lower lip and cheek may be naturally adsorbed to the airway extension device 1 in an embodiment by the negative pressure in the wearer's mouth formed by the above-described negative pressure forming hole 50, the wearer's mouth is prevented from being opened during the sleep to prevent the mouth breathing as well as the nose breathing is induced to maintain the humidity of the entire respiratory system including the oral cavity and the nasal cavity, thereby improving the oral dryness, the bad breath, the rhinitis, the bronchitis and the like.

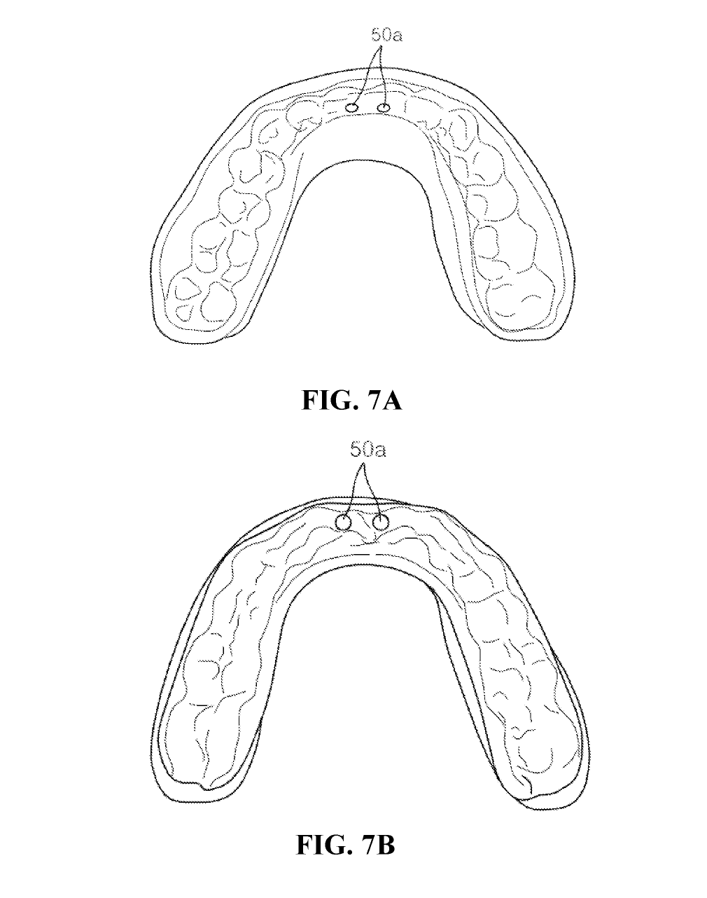

FIGS. 7A, 7B, 8A, 8B, 9A, 9B, 10A and 10B are plan views and bottom views of the airway extension device according to various embodiments disclosed herein.

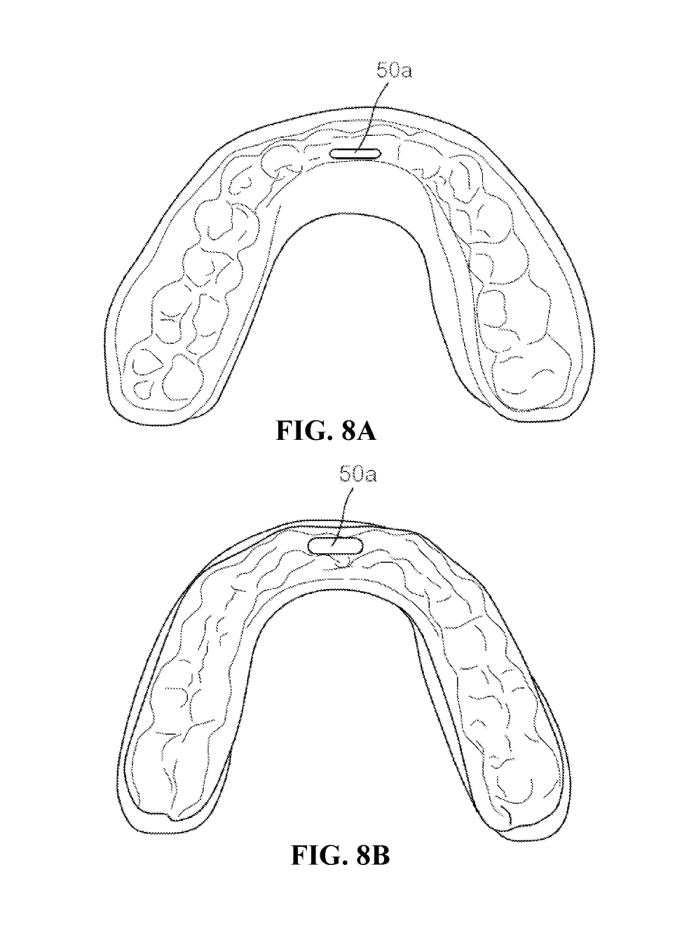

In the airway extension device 1 according to another embodiment disclosed herein described with reference to FIGS. 7A, 7B, 8A and 8B, the negative pressure forming hole 50 may include at least one central hole 50a which is formed at a central part of a plurality of upper teeth and a plurality of lower teeth.

Referring to FIGS. 8A and 8B, the central hole 50a may be formed to be long in the lateral direction so that at least two to four of the upper teeth have a left and right length while abutting to each other. Referring to FIGS. 7A and 7B, two central holes 50a are holes having a diameter not exceeding a left and right width of one upper tooth and may be spaced apart from each other.

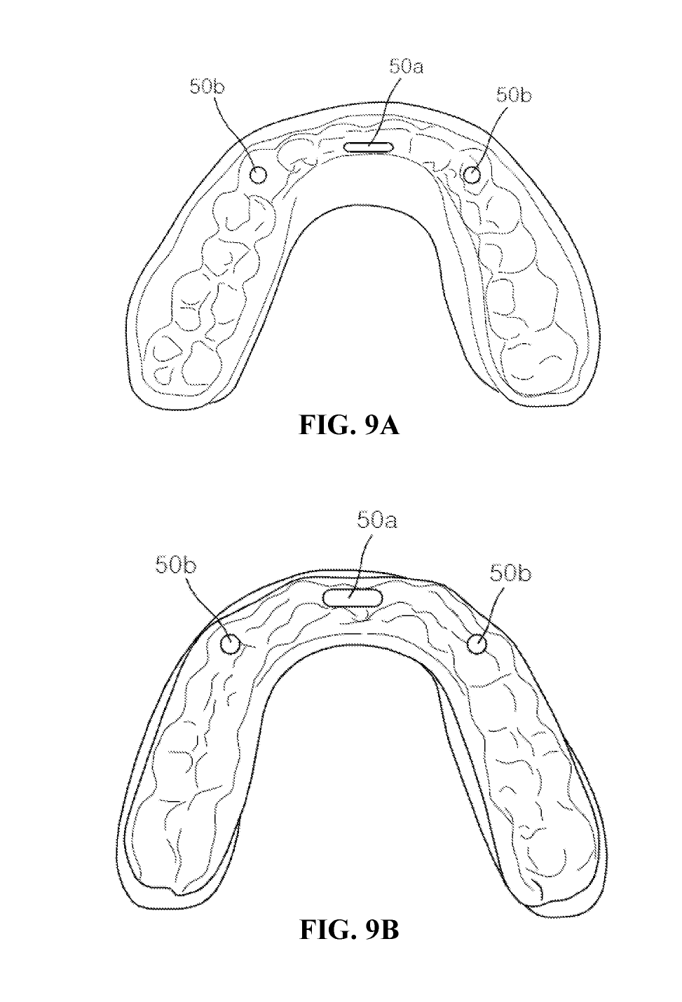

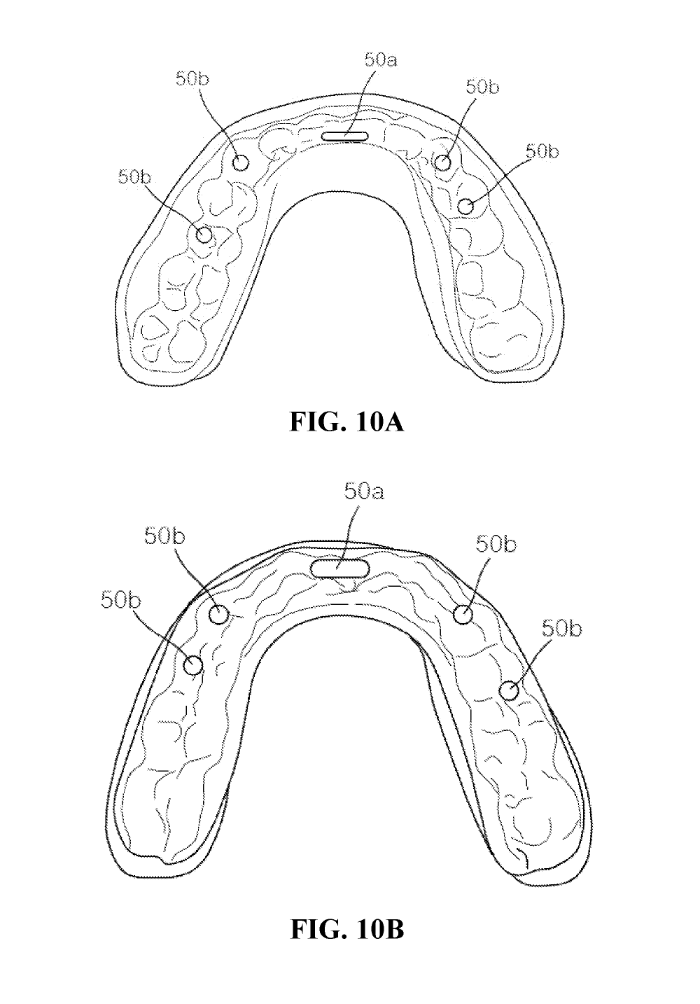

In addition, in the airway extension device 1 according to another embodiment of the present disclosure described with reference to FIGS. 9A, 9B, 10A and 10B, the negative pressure forming hole 50 may further include at least one central hole 50a and at least one side hole 50b each symmetrically formed on the left and right sides of the at least one central hole 50a.

In particular, referring to FIGS. 9A and 9B, in the airway extension device 1 according to another embodiment of the present disclosure, a side hole 50b may be formed at locations spaced by a predetermined distance of the left and right from the central hole 50a one by one.

In addition, referring to FIGS. 10A and 10B, in the airway extension device 1 according to another embodiment of the present disclosure, the side hole 50b may be formed at locations spaced by a predetermined distance of the left and right from the central hole 50a two by two.

The side holes 50b described above may be more advantageous in forming the negative pressure in the oral cavity by further increasing the movement of the wearer's tongue in the sleep state due to the nervous reaction of the tongue in addition to the original central hole 50a.

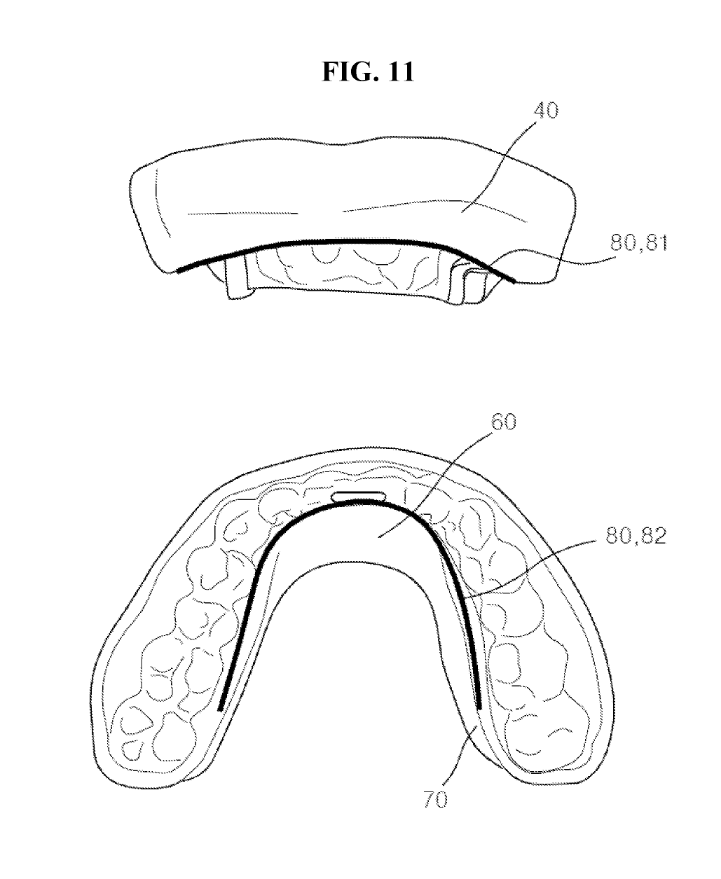

FIG. 11 is a configuration view showing a sealing portion of the airway extension device according to the embodiment of the present disclosure.

Referring to FIG. 11, the airway extension device 1 according to the embodiment of the present disclosure may further include a sealing portion 80 provided in a part of a portion of the airway extension device 1 to avoid a direct contact with the inside of the wearer's oral cavity.

The sealing portion 80 may include a first sealing portion 81 provided on the edge portion to which the wearer's lower lip contacts and a second sealing portion 82 provided on an edge part of the tongue seating surface 60 described above on which the wearer's tongue part is seated.

In more detail, as shown in FIG. 11, the first sealing portion 81 may be provided on the lower edge part of the front surface part of the front rim portion 40 to protect the wearer's lower lip, which is the wearer's soft skin, from being scratched or wounded.

Similarly, as shown in FIG. 11, the second sealing portion 82 may be provided the upper edge of the tongue seating surface 60 and the edge part occupying the left and right side rim portions 70 to protect the tongue, which is a soft skin within the wearer's oral cavity, from being scratched or wounded.

As described above, according to the airway extension device 1 according to the embodiments of the present disclosure, the movement of the tongue within the wearer's oral cavity is guided to the location forming the optimum negative pressure (i.e., location formed at which the negative pressure enough to extend the air passage (airway)) by the at least one negative pressure forming hole 50 provided to penetrate in the vertical direction, thereby improving the snoring or the sleep apnea of the wearer.

The spirit of the present disclosure has been just exemplified. It will be appreciated by those skilled in the art that various modifications and alterations can be made without departing from the essential characteristics of the present disclosure.

According to the airway extension device according to the embodiments of the present disclosure, the air passage (airway) can be secured by compressing the wearer's tongue to the front side during the sleep, thereby improving the snoring, the sleep apnea, the teeth grinding, the clenching or the like.

Further, according to the airway extension device according to the embodiment of the present disclosure, the lower lip and the cheek can be adsorbed on the airway extension device to prevent the mouth from being open during the sleep to prevent the mouth breathing and induce the nose breathing, thereby maintaining the humidity of the respiratory tract including the oral cavity and the nasal cavity to improve the xerostomia, the bad breath, the rhinitis, the bronchitis or the like.

Accordingly, the embodiments disclosed in the present disclosure and the accompanying drawings are used not to limit but to describe the spirit of the present disclosure. The scope of the present disclosure is not limited only to the embodiments and the accompanying drawings. The scope of the present disclosure should be interpreted by the following claims and it should be interpreted that all spirits equivalent to the following claims fall within the scope of the present disclosure.

1. An airway extension device, comprising:
an upper teeth seating portion located on an upper side and seated with a plurality of upper teeth;
a lower teeth seating portion located on a lower side and seated with a plurality of lower teeth; and
at least one negative pressure forming hole formed on a spaced part of the airway extension device corresponding to a spaced location between upper and lower teeth as a location at which the negative pressure is generated when the airway extension device that a wearer wears is mounted in an oral cavity so as to penetrate in a vertical direction.

2. An airway extension device, comprising:
an upper teeth seating portion located on an upper side and seated with a plurality of upper teeth;
a lower teeth seating portion located on a lower side and seated with a plurality of lower teeth;
a teeth partition surface formed to partition the upper teeth seating portion and the lower teeth seating portion; and
at least one negative pressure forming hole formed on the teeth partition surface and formed on a spaced part corresponding to a spaced location between upper and lower teeth generated after the airway extension device that a wearer wears is mounted in an oral cavity to penetrate in a vertical direction.

3. The airway extension device of claim 1, wherein the at least one negative pressure forming hole is formed between an upper line of tooth point formed by connecting lower ends of the plurality of upper teeth and a lower line of tooth point formed by connecting upper ends of the plurality of lower teeth.

4. The airway extension device of claim 1, further comprising:
a front rim portion provided on a front end side of the upper teeth seating portion and comprising an inner side surface surrounding an outer side surface of the plurality of upper teeth and an outer side surface provided to support an inner side surface of an upper lip.

5. The airway extension device of claim 4, wherein the at least one negative pressure forming hole forms a negative pressure of a predetermined pressure between a tongue located inside an oral cavity and a lower lip located outside the oral cavity, comprising the front rim portion interposed therebetween.

6. The airway extension device of claim 4, wherein the at least one negative pressure forming hole forms a negative pressure at a pressure enough to adhere a tongue located inside an oral cavity comprising the front rim portion interposed therebetween to a palate.

7. The airway extension device of claim 1, wherein the negative pressure forming hole includes at least one central hole which is formed at a central side of the plurality of upper teeth and the plurality of lower teeth.

8. The airway extension device of claim 7, wherein the negative pressure forming hole further includes at least one side hole each symmetrically formed on left and right sides of the at least one central hole.

9. The airway extension device of claim 1, further comprising:
a tongue seating surface seated with an edge part of a tongue while forming an inner side surface of the lower teeth seating portion and partitioning between the plurality of lower teeth,
wherein the at least one negative pressure forming hole is formed at a boundary of the tongue seating surface adjacent to the upper teeth seating portion.

10. The airway extension device of claim 9, further comprising:
left and right side rim portions formed on left and right sides of a tongue seating surface while protruding upward to be partitioned from the lower end part of the upper teeth provided on a side part among the plurality of upper teeth,
wherein the at least one negative pressure forming hole is formed under upper ends of the left and right side rim portions

11. The airway extension device of claim 1, wherein the at least one negative pressure forming hole is provided to be closed at an inside and an outside of the oval cavity due to the nature of a nervous reaction of the tongue or the lower lip and is formed on a location where the lower surface of the tip edge part of the tongue located inside the oval cavity closes the negative pressure forming hole while covering the upper side thereof to penetrate in the vertical direction.

12. The airway extension device of claim 1, further comprising:
a sealing portion provided on a part of a portion contacting an inside of a wearer's oval cavity to block a direct contact with the inside of the oval cavity.

13. The airway extension device of claim 12, comprising:
the sealing portion includes: a first sealing portion provided on an edge end contacting the wearer's lower lip; and
a second sealing portion provided on an edge part of a tongue seating surface on which the wearer's tongue part is seated.
